(19) United States
(12) Patent Application Publication
(10) Pub. No.: US 2026/0355187 A1
(43) Pub. Date: Nov. 12, 2026

(54) LURBINECTEDIN AND DOXORUBICIN COMBINATION

(71) Applicant: PHARMA MAR, S.A., Madrid (ES)

(72) Inventor: Gregory M. Cote, Boston, MA (US)

(21) Appl. No.: 19/129,420

(22) PCT Filed: Nov. 14, 2023
(86) PCT No.: PCT/EP2023/081787

§ 371 Date: May 13, 2025

Related U.S. Application Data
(60) Provisional application No. 63/424,987, filed on Nov. 14, 2022.

(51) Int. Cl. A61K 31/4995; A61K 9/00; A61K 31/704; A61K 38/19; A61K 47/02; A61K 47/12; A61K 47/26; A61P 35/00

(52) U.S. Cl. CPC A61K 31/4995; A61K 9/0019; A61K 31/704; A61K 38/193; A61K 47/02; A61K 47/12; A61K 47/26; A61P 35/00

(57) ABSTRACT

Described are combination therapy for the treatment of cancer, particularly soft-tissue sarcoma, comprising lurbinectedin and doxorubicin at low doses.

### FIELD OF THE INVENTION

The present invention relates to therapeutic treatment of cancers, particularly dosing schedules useful in the treatment of cancer. In particular, the present invention relates to combination therapy using lurbinectedin and doxorubicin at low doses.

### BACKGROUND OF THE INVENTION

Lurbinectedin, also known as PM01183 and initially called tryptamicidin, is a synthetic alkaloid with antineoplastic activity, and the subject of WO 03/01427. Lurbinectedin is a selective inhibitor of oncogenic transcription, induces DNA double-strand break generating apoptosis, and modulates the tumour microenvironment. For example, by inhibiting active transcription in tumour-associates macrophages, lurbinectedin downregulates IL-6, IL-8, CCL2, and VEGF.

The chemical structure of lurbinectedin is represented as follows:

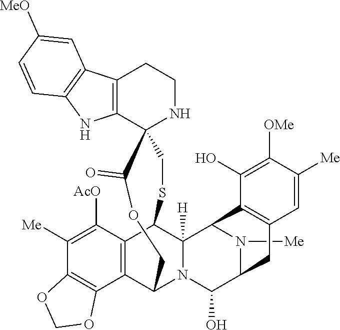 [SMILES: COc1ccc2[nH]c3c(c2c1)CCN[C@]31CS[C@@H]2c3c(OC(C)=O)c(C)c4c(c3[C@H](COC1=O)N1[C@@H]2[C@H]2c3c(cc(C)c(OC)c3O)C[C@@H]([C@@H]1O)N2C)OCO4]

It is a selective inhibitor of the oncogenic transcription programs on which many tumours are particularly dependent. Together with its effect on cancer cells, lurbinectedin inhibits oncogenic transcription in tumour-associated macrophages, downregulating the production of cytokines that are essential for the growth of the tumour. Transcriptional addiction is an acknowledged target in those diseases, many of them lacking other actionable targets.

Lurbinectedin received US approval in 2020 and is marketed in the US for the treatment of adult patients with metastatic small cell lung cancer (SCLC) with disease progression on or after platinum-based chemotherapy. The recommended dosage is 3.2 mg/m2 every 21 days by intravenous infusion. In 2021, lurbinectedin also received marketing authorization in the United Arab Emirates, Canada, Australia and Singapore.

Doxorubicin is a cytotoxic, anthracycline, topoisomerase II inhibitor isolated from cultures of Streptomyces peucetius var. caesius. Chemically, doxorubicin hydrochloride is: 5,12-Naphthacenedione, 10-[(3-amino-2,3,6-trideoxy-α-L-lyxo-hexopyranosyl)oxy]-7,8,9,10tetrahydro-6,8,11-trihydroxy-8-(hydroxylacetyl)-1-methoxy-, hydrochloride (8S-cis).

The cytotoxic effect of doxorubicin on malignant cells is related to nucleotide base intercalation and cell membrane lipid binding activities of doxorubicin. The interaction of doxorubicin with topoisomerase II to form DNA-cleavable complexes is an important mechanism of doxorubicin HCl cytocidal activity.

Doxorubicin is FDA approved and marketed for use in the treatment of acute lymphoblastic leukemia, acute myeloblastic leukemia, Hodgkin lymphoma, non-Hodgkin lymphoma, metastatic breast cancer, metastatic Wilms' tumor, metastatic neuroblastoma, metastatic soft tissue sarcoma, metastatic bone sarcomas, metastatic ovarian carcinoma, metastatic transitional cell bladder carcinoma, metastatic thyroid carcinoma, metastatic gastric carcinoma, and metastatic bronchogenic carcinoma. As a single agent, the recommended standard starting dose of doxorubicin per cycle in adults is 60-75 mg/m2 of body surface area, with standard doxorubicin administration cycles being limited to 6 cycles.

Different clinical trials have been carried out on the combination of lurbinectedin and doxorubicin:

Closed study Phase I Multicenter, Open-label, Clinical and Pharmacokinetic Study of PM01183 in Combination with Fixed Doxorubicin (DOX) in Non-Heavily Pretreated Patients with Selected Advanced Solid Tumors (PM1183-A-003-10; NCT01970540) determined the recommended dose (RD) of lurbinectedin as 1-hour infusion combined with doxorubicin (DOX) both on day 1 every 3 weeks (D1 q3wk), in patients with selected advanced solid tumors. Lurbinectedin was administered at a dose of 2.0 mg and doxorubicin 50.0 mg/m² every three weeks (q3wk).

In the other hand, completed Phase III study PM1183-C-003-14 ([ATLANTIS]), compared lurbinectedin in combination with doxorubicin vs. CAV or topotecan in patients with small cell lung cancer (SCLC) previously treated with one prior platinum-containing line but no more than one prior chemotherapy-containing line. Patients received a dose of 40 mg/m² of doxorubicin and a dose 2.0 mg/m² of lurbinectedin by intravenous infusion (i.v) on D1 q3wk for up to ten cycles, followed by single-agent lurbinectedin 3.2 mg/m² i.v. on D1 q3wk.

In relation with the treatment of sarcomas, Cote et al. Eur, J. Cancer 126 (2020) p. 21-32 disclosed a phase 2 multi-strata study using a combination of doxorubicin and lurbinectedin for the treatment of metastatic and/or resectable sarcomas, for example leiomyosarcoma (LMS). Patients received 50 mg/m2 of doxorubicin followed by 2 mg/m² of lurbinectedin on day 1 of a 21-day cycle. After 6 cycles, patients with disease control continued with lurbinectedin alone at 3.2 mg/m².

There is a continuing need for more effective cancer therapies which are safe and efficacious.

### SUMMARY OF THE INVENTION

The present inventors have determined dosing regimens of combination of lurbinectedin and doxorubicin in the treatment of cancer.

In an aspect, there is provided lurbinectedin for use in the treatment of cancer in a patient need thereof, wherein lurbinectedin is administered in combination with doxorubicin; and wherein doxorubicin is administered at a dose of 30 mg/m² or less during each administration cycle.

In a further aspect, there is provided lurbinectedin for use in the treatment of cancer in a patient need thereof, wherein lurbinectedin is administered in combination with doxorubicin; and wherein doxorubicin is administered as a low dose.

In a further aspect, there is provided lurbinectedin for use in the treatment of cancer in a patient need thereof, said treatment comprises:
i) administering to the patient one or more cycles of lurbinectedin in combination with doxorubicin in a first phase, wherein doxorubicin is administered as low dose, and
ii) administering to the patient one or more cycles of lurbinectedin alone in a second phase.

In a further aspect, there is provided lurbinectedin for use in the treatment of cancer in a patient need thereof, said treatment comprises:
i) administering to the patient one or more cycles of lurbinectedin in combination with doxorubicin in a first phase, wherein doxorubicin is administered at a dose of 30 mg/m² or less during each administration cycle, and
ii) administering to the patient one or more cycles of lurbinectedin alone in a second phase.

In a further aspect, there is provided doxorubicin for use in the treatment of cancer in a patient in need thereof, wherein said treatment comprises administering to the patient lurbinectedin in combination with doxorubicin; and wherein doxorubicin is administered as a low dose; or wherein doxorubicin is administered at a dose of 30 mg/m² or less during each administration cycle.

In a further aspect, there is provided lurbinectedin and doxorubicin for use in the treatment of cancer in a patient need thereof, wherein said treatment comprises administering to the patient a combination of lurbinectedin and doxorubicin; and wherein doxorubicin is administered as a low dose; or wherein doxorubicin is administered at a dose of 30 mg/m² or less during each administration cycle.

In a further aspect, there is provided doxorubicin for use in the treatment of cancer in a patient need thereof, said treatment comprises:
i) administering to the patient one or more cycles of doxorubicin in combination with lurbinectedin in a first phase; wherein: doxorubicin is administered as low dose, or wherein doxorubicin is administered at a dose of 30 mg/m² or less during each administration cycle; and
ii) administering to the patient one or more cycles of lurbinectedin alone in a second phase.

In a further aspect, there is provided lurbinectedin and doxorubicin for use in the treatment of cancer in a patient need thereof, said treatment comprises:
i) administering to the patient one or more cycles of lurbinectedin in combination with doxorubicin in a first phase; wherein doxorubicin is administered as low dose, or wherein doxorubicin is administered at a dose of 30 mg/m² or less during each administration cycle; and
ii) administering lurbinectedin to the patient in a second administration cycle.

In a further aspect, there is provided a pharmaceutical package comprising lurbinectedin, together with instructions for its use in combination with doxorubicin, wherein doxorubicin is administered as a low dose.

In a further aspect, there is provided a pharmaceutical package comprising lurbinectedin, together with instructions for its use in combination with doxorubicin, wherein doxorubicin is administered at a dose of 30 mg/m² or less during each administration cycle.

In a further aspect, there is provided a pharmaceutical package comprising doxorubicin, together with instructions for its use in combination with lurbinectedin; wherein doxorubicin is administered as a low dose, or wherein doxorubicin is administered at a dose of 30 mg/m² or less during each administration cycle.

In a further aspect, there is provided a pharmaceutical package comprising lurbinectedin and doxorubicin, together with instructions for their use in combination, wherein doxorubicin is administered as low dose, or wherein doxorubicin is administered at a dose of 30 mg/m² or less during each administration cycle.

In a further aspect, there is provided a method of treatment of cancer, the method comprising administering a combination therapy of lurbinectedin and doxorubicin to a patient in need thereof, wherein doxorubicin is administered as low dose.

In a further aspect, there is provided a method of treatment of cancer, the method comprising administering a combination therapy of lurbinectedin and doxorubicin to a patient in need thereof, wherein doxorubicin is administered at a dose of 30 mg/m² or less during each administration cycle.

In a further aspect, there is provided a method of treatment of cancer, the method comprising administering to a combination therapy of lurbinectedin and doxorubicin to a patient in need thereof according to the following schedule:
i) administering to the patient one or more cycles of lurbinectedin in combination with doxorubicin in a first phase; wherein doxorubicin is administered as a low dose, or at a dose of 30 mg/m² or less during each administration cycle, and
ii) administering to the patient one or more cycles of lurbinectedin alone in a second phase.

In a further aspect, there is provided the use of lurbinectedin in the manufacture of a medicament for the treatment of cancer, wherein said treatment comprises administering a combination therapy of lurbinectedin and doxorubicin to a patient in need thereof, wherein doxorubicin is administered as low dose; or wherein doxorubicin is administered at a dose of 30 mg/m² or less during each administration cycle.

In a further aspect, there is provided the use of doxorubicin in the manufacture of a medicament for the treatment of cancer, wherein said treatment comprises administering a combination therapy of lurbinectedin and doxorubicin to a patient in need thereof, wherein doxorubicin is administered as low dose; or wherein doxorubicin is administered at a dose of 30 mg/m² or less during each administration cycle.

In a further aspect there is provided the use of lurbinectedin and doxorubicin in the manufacture of a medicament for the treatment of cancer, wherein said treatment comprises administering a combination therapy of lurbinectedin and doxorubicin to a patient in need thereof, wherein doxorubicin is administered as low dose; or wherein doxorubicin is administered at a dose of 30 mg/m² or less during each administration cycle.

In a further aspect, there is provided the use of lurbinectedin in the manufacture of a medicament for the treatment of cancer, wherein said treatment comprises:
i) administering to the patient one or more cycles of lurbinectedin in combination with doxorubicin in a first phase; wherein doxorubicin is administered as a low dose, or wherein doxorubicin is administered at a dose of 30 mg/m² or less during each administration cycle, and
ii) administering to the patient one or more cycles of lurbinectedin alone in a second phase.

In a further aspect, there is provided the use of doxorubicin in the manufacture of a medicament for the treatment of cancer, wherein said treatment comprises:
i) administering to the patient one or more cycles of lurbinectedin in combination with doxorubicin in a first phase; wherein doxorubicin is administered as a low dose, or wherein doxorubicin is administered at a dose of 30 mg/m² or less during each administration cycle, and
ii) administering to the patient one or more cycles of lurbinectedin alone in a second phase.

In a further aspect, there is provided the use of lurbinectedin and doxorubicin in the manufacture of a medicament for the treatment of cancer, wherein said treatment comprises:
i) administering to the patient one or more cycles of lurbinectedin in combination with doxorubicin in a first phase; wherein doxorubicin is administered as a low dose, or wherein doxorubicin is administered at a dose of 30 mg/m² or less during each administration cycle, and
ii) administering to the patient one or more cycles of lurbinectedin alone in a second phase.

The following embodiments apply to all aspects of the present invention.

In an embodiment of the invention, doxorubicin may be administered at a dose of 30 mg/m² or less during each administration cycle.

In an embodiment of the invention, doxorubicin may be administered at a dose of less than 30 mg/m² during each administration cycle.

In a further embodiment, doxorubicin may be administered at a dose between 15 to 30 mg/m² during each administration cycle.

In a further embodiment, doxorubicin may be administered at a dose between 20 to 30 mg/m² during each administration cycle.

In a further embodiment, doxorubicin may be administered at a dose between 15 to less than 30 mg/m² during each administration cycle.

In a further embodiment, doxorubicin may be administered at a dose between 20 to less than 30 mg/m² during each administration cycle.

In a further embodiment, doxorubicin may be administered at a dose between 15 to 25 mg/m² during each administration cycle.

In a further embodiment, doxorubicin may be administered at a dose between 20 to 25 mg/m² during each administration cycle.

In a further embodiment, doxorubicin may be administered at a dose of about 25 mg/m² during each administration cycle.

In a further embodiment, doxorubicin may be administered at a dose of 25 mg/m² during each administration cycle.

In a further embodiment, a single dose of doxorubicin is administered at each administration cycle.

In a further embodiment, a single dose of doxorubicin is administered on day 1 of each administration cycle.

In a further embodiment, a single dose of about 25 mg/m² of doxorubicin is administered at each administration cycle.

In a further embodiment, a single dose of 25 mg/m² of doxorubicin is administered at each administration cycle.

In a further embodiment, lurbinectedin may be administered at a dose between 2 mg/m² and 4 mg/m2 body surface area during each administration cycle.

In a further embodiment, lurbinectedin may be administered at a dose higher than 2 mg/m² and lower than 4 mg/m² body surface area during each administration cycle.

In a further embodiment, lurbinectedin may be administered at a dose between 2.5 and 3.5 mg/m² body surface area during each administration cycle.

In a further embodiment of the invention, lurbinectedin may be administered on day 1 (D1) of each administration cycle.

In a further embodiment, lurbinectedin may be administered at a dose of about 3.2 mg/m² body surface area during each administration cycle.

In a further embodiment, lurbinectedin may be administered at a dose of 3.2 mg/m² body surface area during each administration cycle.

In a further embodiment, lurbinectedin may be administered on day 1 (D1) of each administration cycle.

In a further embodiment, lurbinectedin may be administered at a dose of about 3.2 mg/m² body surface area during each administration cycle; and doxorubicin may be administered as: a low dose, at a dose of 30 mg/m² or less during each administration cycle, at a dose between 15 to 30 mg/m² during each administration cycle, at a dose between 20 to 30 mg/m² during each administration cycle, at a dose between 15 to 25 mg/m² during each administration cycle, at a dose between 20 to 25 mg/m² during each administration cycle, at a dose of about 25 mg/m² during each administration cycle, or a dose of 25 mg/m² during each administration cycle.

In a further embodiment, the method further comprises administration of granulocyte-colony stimulating factor (G-CSF).

In a further embodiment, G-CSF may be administered on day 1 of an administration cycle.

In a further embodiment, a patient may receive primary prophylaxis with G-CSF starting 24-72 hours after day 1 of an administration cycle. Administration may be for five days.

In a further embodiment, G-CSF may be administered during one or more subsequent administration cycles.

In a further embodiment, each administration cycle is between 3 and four weeks.

In a further embodiment, each administration cycle is 21 consecutive days.

In a further embodiment, lurbinectedin may be administered on day 1 (D1) of each administration cycle.

In a further embodiment, lurbinectedin and doxorubicin are administered on day 1 (D1) of each administration cycle.

In a further embodiment, lurbinectedin may be administered at a dose of about 3.2 mg/m² body surface area on day 1 (D1) of each administration cycle; and doxorubicin may be administered on day 1 (D1) of each administration cycle: at a low dose, at a dose of 30 mg/m² or less, at a dose between 15 to 30 mg/m², at a dose between 20 to 30 mg/m², at a dose between 15 to 25 mg/m², at a dose between 20 to 25 mg/m², at a dose of about 25 mg/m², or a dose of 25 mg/m².

In a further embodiment, lurbinectedin may be administered at a dose of about 3.2 mg/m² body surface area on day 1 (D1) of each administration cycle; and doxorubicin may be administered on day 1 (D1) of each administration cycle: at a low dose, at a dose of 30 mg/m² or less, at a dose between 15 to 30 mg/m², at a dose between 20 to 30 mg/m², at a dose between 15 to 25 mg/m², at a dose between 20 to 25 mg/m², at a dose of about 25 mg/m², or a dose of 25 mg/m²; wherein each administration cycle is between 3 to 4 weeks.

In a further embodiment, lurbinectedin may be administered at a dose of about 3.2 mg/m² body surface area on day 1 (D1) of each administration cycle; and doxorubicin may be administered on day 1 (D1) of each administration cycle: at a low dose, at a dose of 30 mg/m² or less, at a dose between 15 to 30 mg/m², at a dose between 20 to 30 mg/m², at a dose between 15 to 25 mg/m², at a dose between 20 to 25 mg/m², at a dose of about 25 mg/m², or a dose of 25 mg/m²; wherein each administration cycle is 21 days.

In a further embodiment, lurbinectedin may be administered at a dose of about 3.2 mg/m² body surface area on day 1 (D1) of each administration cycle; and doxorubicin may be administered on day 1 (D1) of each administration cycle: at a low dose, at a dose of 30 mg/m² or less, at a dose between 15 to 30 mg/m², at a dose between 20 to 30 mg/m², at a dose between 15 to 25 mg/m², at a dose between 20 to 25 mg/m², at a dose of about 25 mg/m², or a dose of 25 mg/m²; wherein the method further comprises administration of granulocyte-colony stimulating factor (G-CSF); wherein G-CSF may be administered on day 1 of an administration cycle or wherein a patient may receive primary prophylaxis with G-CSF starting 24-72 hours after day 1 of an administration cycle, and for five days; optionally wherein G-CSF is administered during one or more subsequent administration cycles; optionally wherein each administration cycle is between 3 to 4 weeks or is 21 days.

In a further embodiment, 1, 2, 3, 4, 5, 6, 7, 8, 9, 10, 11, 12, 13, 14, 15, 16, 17, or 18 administration cycles are administered.

In a further embodiment, 2, 3, 4, 5, 6, 7, 8, 9, 10, 11, 12, 13, 14, 15, 16, 17, 18 administration cycles are administered.

In a further embodiment, the patient may have a lifetime doxorubicin limit of 450 mg/m².

In a further embodiment, 7 or more cycles are administered. In a further embodiment 8 or more cycles are administered. In a further embodiment 9 or more cycles are administered. In a further embodiment 10 or more cycles are administered. In a further embodiment 11 or more cycles are administered. In a further embodiment 12 or more cycles are administered. In a further embodiment 13 or more cycles are administered. In a further embodiment 14 or more cycles are administered. In a further embodiment 15 or more cycles are administered. In a further embodiment 16 or more cycles are administered. In a further embodiment 17 or more cycles are administered.

In a further embodiment, 7 to 18 cycles are administered. In a further embodiment, 8 to 18 cycles are administered. In a further embodiment, 9 to 18 cycles are administered. In a further embodiment, 10 to 18 cycles are administered. In a further embodiment, 11 to 18 cycles are administered. In a further embodiment, 12 to 18 cycles are administered. In a further embodiment, 13 to 18 cycles are administered. In a further embodiment, 14 to 18 cycles are administered. In a further embodiment, 15 to 18 cycles are administered. In a further embodiment, 16 to 18 cycles are administered. In a further embodiment, 17 to 18 cycles are administered. In a further embodiment 18 cycles are administered.

In a further embodiment, lurbinectedin and doxorubicin may be administered as 1 hour intravenous infusion during each administration cycle.

In a further embodiment, doxorubicin is administered first, followed by lurbinectedin administration.

In a further embodiment, after administration of the combination of lurbinectedin and doxorubicin for one or more cycles during a first phase; lurbinectedin is administered alone for one or more cycles during a second phase.

### BRIEF DESCRIPTION OF THE FIGURES

FIG. 1 shows best response by RECIST 1.1 as progressive disease; stable disease; and partial response in 10 patients with different sarcomas: leiomyosarcoma (LMS including uterine leiomyosarcoma (uLMS)), dedifferentiated liposarcoma (DDLPS), undifferentiated pleomorphic sarcoma (UPS), solitary fibrous tumor (SFT), endometrial stromal sarcoma (ESS), and myxofibrosarcoma (myxFS).

FIG. 2 shows the time on study for each patient. PR means partial response; PD means progression disease and the arrow indicates that the treatment is still administered.

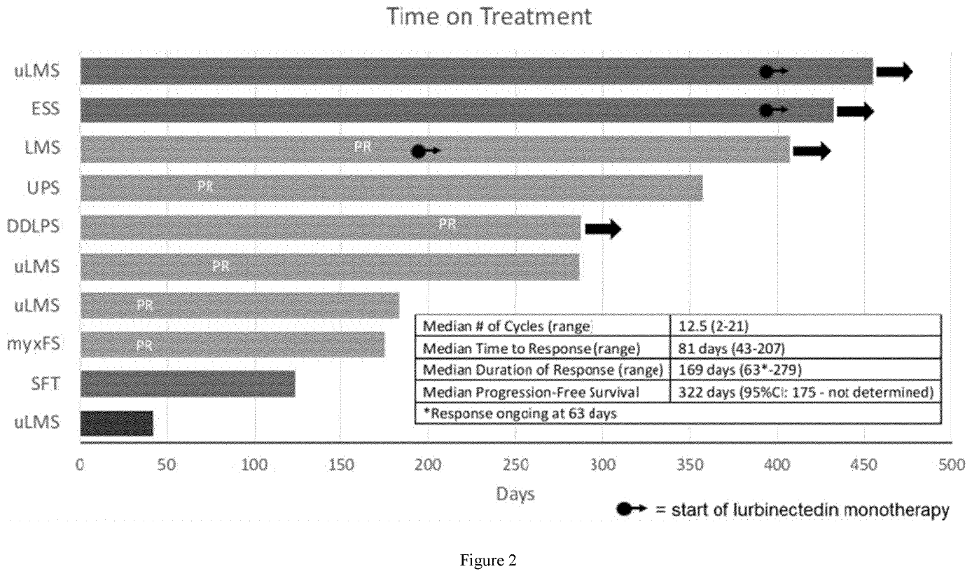

### DETAILED DESCRIPTION OF THE INVENTION

In the present application, a number of general terms and phrases are used, which should be interpreted as follows.

The term “treating” as used herein, unless otherwise indicated, means reversing, attenuating, alleviating or inhibiting the progress of the disease or condition to which such term applies, or one or more symptoms of such disorder or condition. The term “treatment” as used herein, unless otherwise indicated, refers to the act of treating as “treating” is defined immediately above.

“Patient” includes humans, non-human mammals (e.g. dogs, cats, rabbits, cattle, horses, sheep, goats, swine, deer, and the like) and non-mammals (e.g. birds, and the like), preferably humans.

To provide a more concise description, some of the quantitative expressions given herein are not quantified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

The expression “administration cycle” refers to a period of treatment, wherein a single cancer drug or a combination of drugs are administered and usually followed by a period of rest. In the present invention, a treatment cycle is a period of four weeks, preferably three weeks, more preferably 21 consecutive days.

The expression “phase” in the present invention, refers to a period of treatment comprising various administration cycles.

“Lurbinectedin” or PM01183 is a new synthetic alkaloid that which binds the DNA minor groove causing spatial distortion of DNA and protein complexes and leading to the formation of DNA double-strand breaks (DSBs), thus inducing apoptosis and delaying progression through the cell cycle S/G2 phase. Lurbinectedin has the following structure:

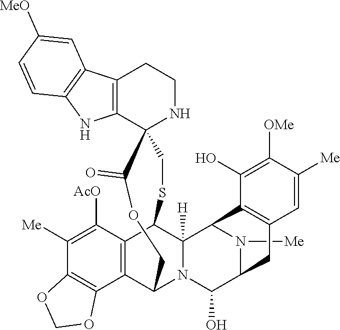 [SMILES: COc1ccc2[nH]c3c(c2c1)CCN[C@]31CS[C@@H]2c3c(OC(C)=O)c(C)c4c(c3[C@H](COC1=O)N1[C@@H]2[C@H]2c3c(cc(C)c(OC)c3O)C[C@@H]([C@@H]1O)N2C)OCO4]

Lurbinectedin has a negative COMPARE analysis when compared against other 98 standard anticancer agents in the standard National Cancer Institute (NCI) panel of 36 cell lines. Thus, its mechanism of action is likely to differ significantly from the other drugs. It only showed a positive correlation (S-rank >0.8) with trabectedin.

In vitro, lurbinectedin demonstrated cytotoxic effects against a broad selection of tumour-derived cell lines with half maximal inhibitory concentration (IC₅₀) values in the low to very low nanomolar range (approximately median IC₅₀ of 1E-10 M).

Further information regarding the clinical development of PM01183 (lurbinectedin) can be found in:
Elez, M E. et. al. Clin. Cancer Res. 2014, 20 (8), 2205-2214;
50^{th} ASCO Annual Meeting, May 30-Jun. 3, 2014, Chicago, IL, Abstract 5505;
26^{th} EORTC-26th EORTC-NCI-AACR Symposium on Molecular Targets and Cancer Therapeutics; Nov. 18-21, 2014, Barcelona, Spain, published in Eur. J. Cancer 2014, 50 (Suppl.6), pages 13-14, Abs. No. 23.
51^{th} ASCO Annual Meeting, May 29-Jun. 2, 2015, Chicago, IL, Abstract No. TPS2604 and Abstract Nr. 7509, published in J. Clin. Oncol. 33, 2015 (suppl);
54th ASCO Annual Meeting, Jun. 1-5, 2018, Chicago, IL, Abstract No. 11519, published in J. Clin. Oncol. 36, 2018 (suppl);
Cruz, C. et al. J. Clin. Oncol. 2018, 36 (31), 3134-3143;
54^{th} ASCO Annual Meeting, Jun. 1-5, 2018, Chicago, IL, Abstract No. 8570, published in J. Clin. Oncol. 36, 2018 (suppl).

Further information may be found in Xie et al Lurbinectedin synergizes with immune checkpoint blockade to generate anticancer immunity, Oncoimmunology, 2019, Vol. 8, No. 11, e1656502 (9 pages).

In addition, any drug referred to herein may be in crystalline or amorphous form either as free compounds or as solvates (e.g. hydrates) and it is intended that all forms are within the scope of the present invention. Methods of solvation are generally known within the art.

The preferred route of administration is parenteral administration including, but not limited to, intradermal, intramuscular, intraperitoneal, intravenous, subcutaneous, intranasal, epidural, intracerebral, intraventricular, intrathecal, intravaginal or transdermal. The preferred mode of administration is left to the discretion of the practitioner, and will depend in part upon the site of the medical condition. In a more preferred embodiment, lurbinectedin and doxorubicin according to the present invention are administered intravenously.

In specific embodiments, it can be desirable to administer lurbinectedin and/or doxorubicin locally to the area in need of treatment. In one embodiment, administration can be by direct injection at the site (or former site) of a cancer, tumour or neoplastic or preneoplastic tissue.

In a particular embodiment, lurbinectedin and/or doxorubicin for use in the treatment of cancer, may be administered by infusion and with an infusion time of up to 24 hours, 1 to 12 hours, 1 to 6 hours and most preferably 1 hour. In embodiments, dosing may be 5 minutes to +20 minutes of the stated infusion time.

Lurbinectedin may be administered in administration cycles every three weeks or every four weeks, preferably every three weeks.

By “low dose” doxorubicin, it means a dose which is lower than a dose which is typically used for doxorubicin. Low dose may mean a dose which is lower than a dose which is currently included in the product information of doxorubicin for a relevant indication.

Reference to a dose in mg/m² refers to a dose based on Body Surface Area (BSA).

In a preferred embodiment, the treatment of the invention comprises:
i) administering to the patient one or more administration cycles of lurbinectedin at a dose of 3.2 mg/m² on D1 of each administration cycle in combination with doxorubicin at a dose of about 25 mg/m² administered on D1 of each administration cycle in a first phase, wherein each administration cycle is 21 days, and
ii) administering to the patient one or more cycles of lurbinectedin alone at a dose of 3.2 mg/m² on D1 of each administration cycle in a second phase.

In a further embodiment, the patient may also receive antiemetic prophylaxis before each infusion, preferably 1 before, more preferably 45 minutes before, more preferably 30 minutes before. Antiemetic prophylaxis comprises corticosteroids and 5HT3 antagonists. Preferably, the corticosteroid is dexamethasone and the 5HT3 antagonist is ondansetron.

The patient may also receive prophylactic medication before getting treatment by infusion as described in the present invention. Prophylactic medication includes corticosteroids and 5-HT3 receptor antagonists. Particular corticosteroids include dexamethasone. Particular 5-HT3 receptor antagonists include ondansetron. Particular dosages include dexamethasone 8 mg i.v. (or an equivalent dose of another i.v. corticosteroid) and ondansetron 8 mg i.v. (or an equivalent dose of another i.v. 5-HT3 receptor antagonist). Prophylactic medication can be administered on Day 1 of each cycle. In addition, further prophylactic medication may be administered as needed. An example includes metoclopramide or equivalent, which in embodiments may be administered every eight hours. After Day 1 and Day 8 of each cycle extended oral corticosteroids (for example dexamethasone not exceeding 20 mg/days) and/or 5-HT3 receptor antagonists (for example oral (or i.v.) ondansetron 4-8 mg (or equivalent)) may be administered.

The patient may also receive granulocyte-colony stimulating factor G-CSF. An example of G-CSF is nonpegylated filgrastim. By way of example, patients may receive primary prophylaxis with G-CSF starting 24-72 hours after day 1 of cycle 1, and during five days. In embodiments, primary G-CSF prophylaxis for further cycles may also be administered using the same regimen. G-CSF prophylaxis may also be administered according to physician discretion.

The present invention has identified dosing regimens useful in the treatment of cancer.

In an embodiment, the cancer is sarcoma.

In an embodiment, the cancer is soft-tissue sarcoma.

Sarcomas are rare cancers that develop in the muscle, bone, nerves, cartilage, tendons, blood vessels and the fatty and fibrous tissues. They can affect almost any part of the body, on the inside or the outside. Sarcomas commonly affect the arms, legs and trunk. They also appear in the stomach and intestines as well as behind the abdomen (retroperitoneal sarcomas) and the female reproductive system (gynaecological sarcomas).

“Soft-tissue sarcoma” can affect any part of the body. They develop in supporting or connective tissue such as the muscle, nerves, fatty tissue, and blood vessels. Soft tissue sarcomas include: GIST which is a common type of sarcoma which develops in the gastrointestinal (G1) tract; gynaecological sarcomas which occur in the female reproductive system: the uterus (womb), ovaries, vagina, vulva and fallopian tubes; and retroperitoneal sarcomas which occur in the retroperitoneum.

There are more than 50 different types of soft tissue sarcomas, including:

Leiomyosarcoma is a type of cancer that starts in smooth muscle tissue. These tumours often start in the abdomen, but they can also start in other parts of the body, such as the arms or legs, or in the uterus.

Liposarcomas are malignant tumours of fat tissue. They can start anywhere in the body, but they most often start in the thigh, behind the knee, and inside the back of the abdomen. They occur mostly in adults between 50 and 65 years old.

Synovial sarcoma is a malignant tumour of the tissue around joints. The most common locations are the hip, knee, ankle, and shoulder. This tumour is more common in children and young adults, but it can occur in older people.

In a preferred embodiment, soft-tissue sarcoma is selected from leiomyosarcoma (LMS), gastrointestinal stromal tumour (GIST), liposarcoma (LPS), dedifferentiated liposarcoma (DDLPS), undifferentiated pleomorphic sarcoma (UPS), malignant peripheral nerve sheath tumors (MPNST), angiosarcoma, hemangioendothelioma (EHE), solitary fibrous tumor (SFT), fibrosarcoma, dermatofibrosarcoma protuberans (DFSP), low-grade fibromyxoid sarcoma (LGFMS), endometrial stromal sarcoma (ESS), myxofibrosarcoma (myxFS), fibromatosis, follicular dendritic cell sarcoma (FDCS), desmoplastic small round cell tumour (DSRC), pleomorphic liposarcoma (PLS), and synovial sarcoma.

In a more preferred embodiment, cancer may be advanced or metastatic leiomyosarcoma.

The leiomyosarcoma may be selected from somatic soft-tissue leiomyosarcoma, cutaneous or subcutaneous leiomyosarcoma, leiomyosarcoma of vascular origin, and leiomyosarcoma of uterine (uLMS) or non-uterine origin.

In a particular embodiment, a single dose of about 25 mg/m² of doxorubicin is administered in combination with lurbinectedin at a dose of about 3.2 mg/m² on D1 of each administration cycle for use in the treatment of leiomyosarcoma.

The pharmaceutical compositions can be prepared using methodology well known in the pharmaceutical art. For example a composition intended to be administered by injection can be prepared by combining lurbinectedin with water, or other physiologically suitable diluent, such as phosphate buffered saline, so as to form a solution. A surfactant can be added to facilitate the formation of a homogeneous solution or suspension.

Preferred compositions comprising lurbinectedin in the invention may include:
Pharmaceutical compositions comprising lurbinectedin and a disaccharide. Particularly preferred disaccharides are selected from lactose, trehalose, sucrose, maltose, isomaltose, cellobiose, isosaccharose, isotrehalose, turanose, melibiose, gentiobiose, and mixtures thereof.
Lyophilised pharmaceutical compositions comprising lurbinectedin and a disaccharide. Particularly preferred disaccharides are selected from lactose, trehalose, sucrose, maltose, isomaltose, cellobiose, isosaccharose, isotrehalose, turanose, melibiose, gentiobiose, and mixtures thereof.

The ratio of lurbinectedin to the disaccharide in embodiments of the present invention is determined according to the solubility of the disaccharide and, when the formulation is freeze dried, also according to the freeze-dryability of the disaccharide. It is envisaged that this lurbinectedin:disaccharide ratio (w/w) can be about 1:10 in some embodiments, about 1:20 in other embodiments, about 1:50 is still further embodiments. It is envisaged that other embodiments have such ratios in the range from about 1:5 to about 1:500, and still further embodiments have such ratios in the range from about 1:10 to about 1:500.

The composition comprising lurbinectedin may be lyophilized. The composition comprising lurbinectedin is usually presented in a vial which contains a specified amount of such compound.

Lurbinectedin may be a lyophilized powder for concentrate for solution for infusion, as 4 mg/vial. Before use, the 4-mg vial may be reconstituted with 8 mL of sterile water for injection, to give a solution containing 0.5 mg/mL of lurbinectedin. For administration to patients as i.v. infusion, reconstituted vials may be diluted with glucose 50 mg/mL (5%) or sodium chloride 9 mg/mL (0.9%) solution for infusion.

The full composition of the PM01183 4 mg vial and the reconstituted solution per mL may be as follows

| | | | Concentration per vial | |
|---|---|---|---|---|
| Component | Concentration/vial | | after reconstitution | |
| PM01183 | 4.0 | mg | 0.5 | mg/mL |
| Sucrose | 800 | mg | 100 | mg/mL |
| Lactic acid | 22.08 | mg | 2.76 | mg/mL |
| Sodium hydroxide | 5.12 | mg | 0.64 | mg/mL |

In an embodiment of the invention, a pharmaceutical package comprises lurbinectedin optionally together with instructions for its use in combination with doxorubicin, wherein lurbinectedin is administered at a dose higher than 2 mg/m² and lower than 4 mg/m² on D1 of each administration cycle in combination with doxorubicin at a dose of 30 mg/m² or less administered on D1 of each administration cycle.

In another embodiment of the invention, a pharmaceutical package comprises lurbinectedin and doxorubicin optionally together with instructions for their use in combination, wherein lurbinectedin is administered at a dose higher than 2 mg/m² and lower than 4 mg/m² on D1 of each administration cycle in combination with doxorubicin at a dose of 30 mg/m² or less administered on D1 of each administration cycle.

In a preferred embodiment of the invention, a pharmaceutical package comprises lurbinectedin optionally together with instructions for its use in combination with doxorubicin, wherein lurbinectedin is administered at a dose of 3.2 mg/m² on D1 of each administration cycle in combination with doxorubicin at a dose of about 25 mg/m² administered on D1 of each administration cycle.

The invention will now be described further with reference to the following example:

### EXAMPLE

Phase 1b lead-in to a randomized phase trial 2 of lurbinectedin plus doxorubicin in leiomyosarcoma

### Study Objectives

The objective of Phase 1b study is exploring the safety and efficacy of lurbinectedin with doxorubicin. Upon determination of the maximum tolerated dose (MTD) and recommended Phase 2 dose (RP2D), the randomized phase 2 trial in participants with advanced leiomyosarcoma (LMS) will commence.

### Phase Ib

### Primary Objective:

To determine the MTD and the RDP2 of lurbinectedin with doxorubicin in patients with advanced soft-tissue sarcoma.

### Secondary Objectives:

To evaluate the preliminar anti-tumor activity of the combination, including the disease control rate (DCR) at 6 and 12 months, and the rates of PFS, overall survival (OS), and objective response (OR) assessed by RECIST version 1.1 criteria.
To evaluate the safety and tolerability of the combination of lurbinectedin with doxorubicin.

### Phase II

### Primary Objective:

To determine the progression-free survival (PFS) rate of lurbinectedin with doxorubicin compared to doxorubicin alone in patients with advanced leiomyosarcoma.

### Secondary Objectives:

To evaluate the anti-tumor activity of the combination, including the disease control rate (DCR) at 6 and 12 months, and the rates of OS, and OR assessed by RECIST version 1.1 criteria.
To evaluate the safety and tolerability of the combination of lurbinectedin with doxorubicin.

### Exploratory Objectives for Both Phases:

Archival tumor, germline DNA, and CtDNA will be collected for correlative studies exploring genomic markers of sensitivity/resistance.

### Study Design

The study will be divided into two parts: phase 1b is being done to determine the maximum tolerated dose (MTD) of lurbinectedin with doxorubicin in patients with soft-tissue sarcoma and determine recommended phase II dose. Phase II is a randomized (1:1) study of lurbinectedin+doxorubicin versus doxorubicin monotherapy in anthracycline-naïve leiomyosarcoma.

### Phase Ib

The Phase 1b lead-in follows a standard 3+3 design. Patients will receive two dose levels of doxorubicin (a first dose level with 25 mg/m² of doxorubicin at day 1 only and a second dose level with 25 mg/m² of doxorubicin at day 1 and day 8), intravenously (i.v.) as a 60-minute infusion followed by PM01183 at a fixed dose of 3.2 mg/m² i.v. asa 1-hour infusion on Day 1 every three weeks (q3wk). A cycle is defined as an interval of three weeks. The Phase 1b lead-in follows a standard 3+3 design where dose escalation will occur if 0/3 or 1/6 patients experience a dose-limiting toxicity (DLT).

### Phase II

50 patients with leiomyosarcoma will be randomized 1:1 for enrollment to one of two treatment arms: Arm1:lurbinectedin+ doxorubicin versus doxorubicin monotherapy. Randomization will be stratified by uterine v. non-uterine origin of leiomyosarcoma. Participants on Arm 1 will receive lurbinectedin and doxorubicin at the RP2D defined during Phase 1b. Participants enrolled to Arm 2 will receive single agent doxorubicin at a dose of 75 mg/m² on Day 1 of each cycle up to the lifetime maximum dose of 450 mg/m².
Post progression

Patients with radiologic disease progressing on Arm2 will be permitted to transition to receive lurbinectedin monotherapy at 3.2 mg/m² on Day 1 of each 21-day cycle.

### Study Population

### Inclusion Criteria

### Phase Ib

1) Patients with locally advanced or metastatic soft-tissue sarcoma and no curative multimodality treatment options available
2) Voluntarily signed and dated written informed consent prior to any specific study procedure.
3) Age >18 years.
4) Eastern Cooperative Oncology Group (ECOG) performance status (PS) score≤2. (Karnofsky ≥60%,)
7) Measurable disease according to RECIST v.1.1. Note: irradiated lesions may qualify as target if progression has been documented.
9) Adequate bone marrow, renal, hepatic, and metabolic function (assessed≤7 days before inclusion in the study):
   a) Platelet count ≥100×109/L, hemoglobin ≥9.0 g/dL and absolute neutrophil count (ANC) ≥1.5×109/L.
   b) Aspartate aminotransferase (AST) and alanine aminotransferase (ALT) ≤3.0×the upper limit of normal (ULN), independently of the presence of liver metastases.
   c) Alkaline phosphatase (AP) ≤2.5×ULN.
   d) Total bilirubin ≤1.5×ULN or direct bilirubin ≤ULN.
   e) International Normalized Ratio (INR) <1.5 (except if patient is on oral anticoagulation therapy).
   f) Calculated creatinine clearance (CrCL) ≥30 mL/minute (using Cockcroft and Gault's formula).
   g) Creatine phosphokinase (CPK) ≤2.5×ULN.
   h) Albumin ≥3.0 g/dL. Albumin infusion to fulfill the inclusion criterion is forbidden.
   i) Thyroid stimulating hormone (TSH) within institutional normal limits. If TSH is above the ULN, then a free T4 within institutional normal limits is acceptable.
10) Evidence of non-childbearing status for women of childbearing potential (WOCBP). Both women and men must agree to use a highly effective contraceptive measure prior to study entry and during the trial and for at least 6 months after completion of study agent administration. Fertile male patients with WOCBP partners must agree to refrain from fathering a child or donating sperm during the trial and up to five months after treatment discontinuation. Acceptable methods of contraception include abstinence, intrauterine device (IUD), oral contraceptive, subdermal implant and/or double barrier.
11) For participants with known chronic hepatitis B virus (HBV) infection, the HBV viral load must be undetectable on suppressive therapy, if indicated.
12) Participants with a known history of hepatitis C virus (HCV) infection must have been treated and cured. For participants with known HCV infection who are currently on treatment, they are eligible if they have an undetectable HCV viral load.
13) Left ventricular ejection fraction (LVEF) ≥50% on screening echocardiogram (ECHO) or multigated acquisition (MUGA) scan.
14) Participants must have archival tissue available for analysis in the form of a formalin-fixed paraffin embedded (FFPE) block or unstained slides. Participants without archival tissue available may be enrolled with approval of the Sponsor-Investigator. Note: confirmation of availability of archival tissue is the only requirement for eligibility, archival tissue does not need to be received by the study team or site prior to enrollment.
15) Patients with a prior or concurrent malignancy whose natural history or treatment does not have the potential to interfere with the safety or efficacy assessment of the investigational regimen as assessed by the treating investigator may be included with the approval of the Sponsor-Investigator.

### Phase II

Participants must have histologically confirmed advanced or metastatic leiomyosarcoma (LMS) and no curative multimodality treatment options available.

### Exclusion Criteria

1) Participants who have received prior anthracycline or trabectedin (Yondelis, ET-743), including prior exposure to doxorubicin or liposomal doxorubicin.
2) Participants who have received more than 2 prior lines of cytotoxic chemotherapy for the phase 1b study and no more than 1 prior line of cytotoxic chemotherapy for the phase 2 study. There is no limit on the number of prior lines of non-cytotoxic chemotherapy (e.g., pazopanib, immunotherapy).
3) Prior exposure to lurbinectedin (PM01183).
4) Participants who have received prior radiation treatment of >45 Gy to the pelvis.
5) Participants who have received or undergone prior chemotherapy within 14 days of cycle 1 day 1, therapeutic radiation therapy within 21 days of cycle 1 day 1 or major surgery within 21 days of cycle 1 day 1.
6) Participants who have received prior palliative radiation therapy within 7 days of cycle 1 day 1.
7 Participants who have received prior antibody-based therapy (e.g., nivolumab) within 4 weeks or 3 half-lives (whichever is shorter) of cycle 1 day 1.
8) Participants who have received prior oral small molecule or tyrosine kinase inhibitor (TKI) therapy within 2 weeks or 3 half-lives (whichever is shorter) of cycle 1 day 1.
9) Participants who have not recovered to ≤Grade 1 or baseline from adverse events attributed to any prior anti-cancer therapy, with the exceptions of alopecia, controlled endocrine toxicity (e.g., hypothyroidism), and cutaneous toxicity which will be permitted at Grade 2.
10) Participants who are receiving any other investigational agents.
11) Participants with known CNS disease involvement, with the exception of patients with brain metastases that have been previously treated and have remained stable on MRI ≥28 days prior to cycle 1 day 1 without use of steroids or anti-epileptic medications.
12) History of allergic reactions attributed to compounds of similar chemical or biologic composition to lurbinectedin or doxorubicin.
13) Participants receiving any medications or substances that are strong or moderate inhibitors or inducers of CYP3A, CYP2D6, or P-gp are ineligible. Because the lists of these agents are constantly changing, it is important to regularly consult a frequently updated medical reference. As part of the enrollment/informed consent procedures, the participant must be counseled on the risk of interactions with other agents, and what to do if new medications need to be prescribed or if the participant is considering a new over-the-counter medicine or herbal product.
14) Uncontrolled intercurrent illness including, but not limited to: ongoing or active infection, symptomatic congestive heart failure, unstable angina pectoris, cardiac arrhythmia, chronic indwelling drains, or psychiatric illness/social situations that would limit compliance with study requirements.
15) History of interstitial pneumonitis or pulmonary fibrosis.
16) Known cardiomyopathy.
17) Pregnant women are excluded from this study because lurbinectedin and doxorubicin are anti-cancer agents with the potential for teratogenic or abortifacient effects. Because there is an unknown but potential risk for adverse events in nursing infants secondary to treatment of the mother with lurbinectedin or doxorubicin, breastfeeding must be discontinued if the mother is treated with lurbinectedin or doxorubicin. A negative pregnancy test is required for women of childbearing potential prior to the first dose of study medication.
18) Immunocompromised patients, including patients who are known to be seropositive for human immunodeficiency virus (HIV) due to the increased risk of lethal infections when treated with marrow-suppressive therapy. HIV testing is not required as part of screening.

### Study Population

### Expected Number of Patients

Phase Ib follows a standard 3+3 design. A minimum of 2 patients and a maximum of 12 patients are enrolled in Phase 1b. The starting dose is Dose Level 1. Three participants may be enrolled at the starting dose level 1. A minimum of 6 patients must be treated at the recommended Phase II dose (R2PD). In Phase II 50 patients, for an overall maximum sample size of 62 participants, will be randomized 1:1 to one of two treatments arms: Arm 1:lurbinectedin+doxorubicin and

Arm 2: doxorubicin monotherapy. Participants enrolling to the Phase 2 will be stratified by uterine versus non-uterine leiomyosarcoma.

### Study Drug

### Formulation

### PM01183

PM01183 drug product is provided as a lyophilized powder for concentrate for solution for infusion in 4-mg vials.

Before use, the 4-mg vial should be reconstituted with 8 mL of sterile water for injection to give a solution containing 0.5 mg/mL of PM01183. For administration to patients as an i.v. infusion, reconstituted vials should be diluted either with glucose 50 mg/mL (5%) solution or sodium chloride 9 mg/mL (0.9%) solution for infusion.

The full composition of the PM01183 4-mg vials and the reconstituted solution per mL is shown in Table 1.

TABLE 1
Composition of PM01183 vials.

| | PM01183 | | Concentration per vial | |
|---|---|---|---|---|
| Component | 4 mg | | after reconstitution | |
| PM01183 | 4.0 | mg | 0.05 | mg/mL |
| Sucrose | 800 | mg | 100 | mg/mL |
| Lactic acid | 22.08 | mg | 2.76 | mg/mL |
| Sodium hydroxide | 5.12 | mg | 0.64 | mg/mL |

### Doxorubicin

Doxorubicin hydrochloride drug product is provided as lyophilized powder for concentrate for solution for infusion in 10 mg, 20 mg, 50 mg, or 150 mg vials.

Before use, doxorubicin hydrochloride should be reconstituted with 0.9% sodium chloride injection, USP to obtain a final concentration pf 2 mg/mL as follows:
5 mL 0.9% Sodium Chloride Injection, USP to reconstitute 10 mg doxorubicin HCl vial
10 mL 0.9% Sodium Chloride Injection, USP to reconstitute 20 mg doxorubicin HCl vial
25 mL 0.9% Sodium Chloride Injection, USP to reconstitute 50 mg doxorubicin HCl vial
75 mL 0.9% Sodium Chloride Injection, USP to reconstitute 150 mg doxorubicin HCl vial

### Treatment Schedule

### Phase 1b

Lurbinectedin PM01183 is administered at a fixed dose of 3.2 mg/m² as a 1-hour (+5 minutes) i.v. infusion, on Day 1 of each administration cycle.

Doxorubicin is administered i.v infusion per institutional standards of practice and/or the FDA package insert. Doxorubicin dosing to continue up to lifetime maximum dose of 450 mg/m² or until the participant meets other treatment discontinuation criteria. Following discontinuation of doxorubicin, participants will continue to receive lurbinectedin as monotherapy at the dose of 3.2 mg/m² on Day 1 of each administration cycle until treatment discontinuation criteria are met.

An administration cycle is defined as 21 consecutive days.

### Administration Route and Dose

### Phase Ib

On days, where both lurbinectedin and doxorubicin are administered, doxorubicin is administered first followed by lurbinectedin. Lurbinectedin infusion starts within 1 hour of when the doxorubicin infusion is completed.

The starting dose level is Dose level 1.

At Dose level 1 (starting dose level): doxorubicin is administered at a dose of 25 mg/m² as a 1-hour i.v. infusion, on Day 1 only, followed by lurbinectedin at a fixed dose of 3.2 mg/m² as a 1-hour (±5 minutes) i.v. infusion.

At Dose level 2: doxorubicin is administered at a dose of 25 mg/m² as a 1-hour i.v. infusion, on Day 1 and Day 8, followed by lurbinectedin at a fixed dose of 3.2 mg/m² as a 1-hour (±5 minutes) i.v. infusion.

### Dose Escalation Scheme (Phase Ib)

Dose escalation will proceed in a standard 3+3 design in accordance with the following criteria:

| Number of Participants | |
|---|---|
| with DLT at a Given Dose | |
| Level | Escalation Decision Rule |
| 0 out of 3 | Enter 3 participants at the next dose level. |
| ≥2 | Dose escalation will be stopped. This dose |
| | level will be declared the maximally |
| | administered dose (highest dose |
| | administered). Three (3) additional |
| | participants will be entered at the next |
| | lowest dose level if only 3 participants |
| | were treated previously at that dose. |
| 1 out of 3 | Enter at least 3 more participants at this |
| | dose level. |
| | If 0 of these 3 participants experience |
| | DLT, proceed to the next dose level. |
| | If 1 or more of this group suffer DLT, |
| | then dose escalation is stopped, and this |
| | dose is declared the maximally |
| | administered dose. Three (3) additional |
| | participants will be entered at the next |
| | lowest dose level if only 3 participants |
| | were treated previously at that dose. |
| ≤1 out of 6 at highest dose | This is generally the maximum tolerated |
| level below the maximally | dose (MTD). At least 6 participants must be |
| administered dose | entered at the MTD. |

During the Phase Ib, body surface area (BSA) will be calculated every cycle according to the DuBois formula. PM01183 dose will be recalculated before a new cycle is started. Doses will be rounded to the first decimal.

### Prophylactic Medication

On Day 1 of each cycle (before lurbinectedin administration), all patients will receive the following prophylactic medication:
Dexamethasone 8 mg i.v. or equivalent, maximum 20 mg/day. If feasible, the medication and dose administered in Cycle 1 should be maintained in Cycle 2.
5-HT3 antagonists, ie. Ondansetron 8 mg i.v. and no more than 16 mg.

Other possible prophylactic medications:
Treatment with 5-HT3 antagonists and/or dexamethasone could be extended orally, i.e with 4 to 8 mg/day for from three to five consecutive days after drug infusions.
Additional antiemetics might be used, if required.

### Allowed Medications/Therapies

Granulocyte Colony stimulating factor (G-GSF): pegylated G-GSF is required for patients receiving combination treatment with lurbinectedin and doxorubicin. Pegylated G-GSF should be administered on Day 2 in the Dose Level 1 and administered on Day 9 in the Dose Level 2.
Therapies for preexisting and treatment-emergent medical conditions, including pain management and local management of mucositis/stomatits.
Blood products and transfusions, as clinically indicated.
Bisphosphonates.
In case of nausea or vomiting, secondary prophylaxis and/or symptomatic treatment for emesis according to American Society of Clinical Oncology (ASCO) guidelines (taking into account the aforementioned limit of corticosteroids per day).
Erythropoietin use according to ASCO guidelines.
Low molecular weight heparin (LMWH) and/or any other anticoagulants, as clinically indicated. Oral anticoagulants must be carefully monitored.
Palliative limited field bone RT (e.g. for pain control outside the thoracic wall).
Megestrol acetate for appetite stimulation.
Contraceptives.

### Prohibited Medications/Therapies

Concomitant administration of any other antineoplastic therapy.
Other investigational agents.
Inmunopressive therapies other than corticosteroids for antiemetic prophylaxis or pain control, or low-dose replacement in patients requiring this approach.
Aprepitant or fosaprepitant or any other NK-1 antagonist or related substance P-antagonists (except for rolapitant).
CYP3A4 inhibitors such as ketoconazole, fluconazole, voriconazole, telithromycin, clarithromycin, erythromycin, nafcillin, aprepitant, fosaprepritant, verapamil, modafinil, nefazodone, or grapefruit juice.
CYP3A enzyme inducers and/or inhibitors (unless strictly necessary and when there is no therapeutic alternative treatments).
Use of any prescription or non-prescription herbal and/or dietary supplements within 14 days prior to the first dose of medication and until 31 days after the last administration of lurbinectedin, unless the Investigators, with the Sponsor agreement, consider it will not interfere with study procedures of patient safety.

### Drug-Drug Interactions

Lurbinectedin

In vitro studies with human microsomes showed that CYP3A4 is the major CYP isoform involved in the metabolism of PM01183, followed by CYP2E1, CYP2D6 and CYP2C9. The estimated contribution of the other CYP isoenzymes to the PM01183 metabolism is considered to be negligible. Coadministration of lurbinectedin with strong or moderate CYP3A inhibitors is strictly prohibited during cycle 1 for participants enrolled to the dose escalation portion of the trial. For all other participants, coadministration of lurbinectedin with strong or moderate CYP3A inhibitors should be avoided when clinically feasible.

Do not co-administer lurbinectedin with aprepitant or any other NK-1 antagonist or related Substance P-antagonists (except rolapitant).
Doxorubicin

Doxorubicin is a major substrate of cytochrome P450 CYP3A4 and CYP2D6, and P-glycoprotein (P-gp). Clinically significant interactions have been reported with inhibitors of CYP3A4, CYP2D6, and/or P-gp (e.g., verapamil), resulting in increased concentration and clinical effect of doxorubicin. Inducers of CYP3A4 (e.g., phenobarbital, phenytoin, St. John's Wort) and P-gp inducers may decrease the concentration of doxorubicin. Therefore, concurrent use of doxorubicin HCl with inhibitors and inducers of CYP3A4, CYP2D6, or P-gp must be avoided.

### Evaluability of Patients

### Phase Ib

An evaluable patient for the primary objective of this phase Ib (i.e., determination of the MTD and RP2D) should have received at least one complete infusion of doxorubicin and PM01183 and be followed for at least one complete cycle (i.e., three weeks=21 days). Patients who are discontinued early or miss/delay doses and/or clinically relevant assessments (i.e., Hematology) will be evaluable if these events are the consequence of treatment-related toxicity (excluding hypersensitivity reactions and/or extravasations).

### Evaluation Criteria

### Primary Endpoint

### Phase Ib

### Determination of MTD and MAD:

The maximally administered dose (MAD) of the study medication will be defined as the dose level where at least two participants develop toxicities fitting a DLT definition.

The dose level immediately below the MAD will be defined as the MTD.

In the situation where none of the dose levels have ≥2 DLTs, the MTD will be the highest dose administered (i.e. Dose Level 2).

If Dose Level 1 is found intolerable (with either 2/3 or ≥2/6 patients experiencing a DLT), the phase 1b trial will be discontinued.

An alternate dose and/or schedule may be considered with a protocol amendment (e.g. lurbinectedin 2 mg/m² and doxorubicin 50 mg/m², a known tolerable dose schedule).

The MTD will be established in a minimum of 6 participants. Upon declaring the MTD, a review of available safety and efficacy data generated during the phase 1b is realize in order to confirm the RP2D of the combination.

### Secondary Endpoints

Safety: patients will be evaluable for safety if they have received at least one infusion of doxorubicin and PM01183. AEs will be graded according to the National Cancer Institute Common Terminology Criteria for Adverse Events (NCI-CTCAE) v.5.
Efficacy: antitumor activity of the combination will be evaluated in terms of:
   Progression-free survival defined as the time from the date of registration to the date of documented progression per RECIST v.1.1 or death (regardless of the cause of death). If the patient receives further antitumor therapy or is lost to follow-up before PD, PFS will be censored at the date of last tumor assessment before the date of subsequent antitumor therapy.
   Duration of response (DoR) will be calculated from the date of first documentation of response per RECIST v.1.1 (complete or partial response, whichever comes first) to the date of documented PD or death. The censoring rules defined above for PFS will be used for DoR.
   Clinical benefit defined as percentage of evaluable patient with complete response, partial response or stable disease lasting ≥3 months, as defined by RECIST v1.1.
   Overall survival (OS): calculated from the date of registration to the date of death (death event) or last contact (in this case, survival will be censored on that date).
   Mid- and long-term survival (OS at 12, 18 and 24 months) will be the Kaplan-Meier estimates of the probability of being alive at these time points.
Pharmacokinetics: PK parameters will be evaluated in plasma by standard non-compartmental methods (compartmental modeling may be performed if appropriate).
Pharmacogenetics: factors that may help to explain individual variability in main PK parameters, the presence or absence of germline mutations or polymorphisms that may be involved in the metabolism and/or transport of PM01183 will be analyzed in leukocyte DNA extracted.
Pharmacogenomics: In order to determine predictive/prognostic markers of response and/or resistance to PM01183 and doxorubicin, tumor samples available at baseline will be evaluated in all patients. In addition, blood samples (Day 1 of every cycle) and on-treatment tumor sample from biopsy (4th to 6th weeks after treatment onset) will be obtained and evaluated for patients consenting to PGx sub-study.

### Results

A total of 10 patients in Phase 1b were treated, including male 3 pts (30%) and female 7 pts (70%) with median age 59 years. The median ECOG PS (range) was 0. Median of prior lines was 0.5.

From the ten patients enrolled: 5 were affected by leiomyosarcoma (1 patient with LMS and 4 patients with uterine leiomyosarcoma (uLMS)), 1 by dedifferentiated liposarcoma (DDLPS), 1 by undifferentiated pleomorphic sarcoma (UPS), 1 by solitary fibrous tumor (SFT), 1 by endometrial stromal sarcoma (ESS), and 1 by myxofibrosarcoma (myxFS).

3 pts were enrolled at the Dose Level 1 (3.2 mg/m² lurbinectedin+doxorubicin 25 mg/m² on Day 1 only), without DLT.

At Dose Level 2 (3.2 mg/m²+doxorubicin 25 mg/m² on Day 1 and Day 8), 1 patient (1/3 patients) experienced DLT (neutropenia grade 3 at day 8 resulting in doxorubicin hold). This DLT was resolved to grade 1 within 7 days.

A 4^{th} patient was enrolled at Dose Level 2 and experienced a DLT (grade 2 alanine aminotransferase elevation (ALT)/aspartate transferase (AST)). This DLT occurred on day 8 resulting in doxorubicin hold and was resolved to grade 1 within 7 days.

Three additional slots have opened for Dose Level 1 confirmation. 1/3 patients experienced grade 3 ALT elevation which resolved to grade 1 within 4 days.

No other grade 3 or 4 adverse effect have occurred. Most treatment related adverse events were grade 1 or 2 nausea, fatigue, reversible cytopenia, alopecia, anemia, dehydration, typical for doxorubicin and lurbinectedin drugs.

The treatment-emergent adverse events are shown in Table 2.

TABLE 2
treatment-emergent adverse events

| Adverse Event | Grade 2 | Grade 3 |
|---|---|---|
| Fatigue | 5 | 0 |
| Anemia | 3 | 1 |
| Nausea | 5 | 0 |
| Neutrophil count decreased | 4 | 1 |
| Alopecia | 3 | 0 |
| Vomiting | 3 | 0 |
| Anorexia | 2 | 0 |
| ALT elevation | 1 | 1 |
| Dehydration | 2 | 0 |

### Updated Results

Updated treatment-emergent adverse events are shown in Table 3.

TABLE 3
updated treatment-emergent adverse events

| Adverse Event | Grade 2 | Grade 3 | Grade 4 |
|---|---|---|---|
| Fatigue | 7 | 0 | 0 |
| Hypertension | 6 | 0 | 0 |
| Infections | 6 | 0 | 0 |
| Nausea | 5 | 0 | 0 |
| White blood cell decreased | 3 | 1 | 0 |
| Neutrophil count decreased | 3 | 1 | 0 |
| Anemia | 2 | 1 | 0 |
| Vomiting | 3 | 0 | 0 |
| Alopecia | 3 | 0 | 0 |
| Anorexia | 2 | 0 | 0 |
| Dyspnea | 2 | 0 | 0 |
| Alanine aminotransferase increased | 1 | 1 | 0 |
| Dehydration | 2 | 0 | 0 |
| Lymphocyte count decreased | 0 | 1 | 1 |
| Duodenal obstruction | 0 | 1 | 0 |
| Gastric outlet obstruction | 0 | 1 | 0 |

* G2 events with n ≥ 2 included

### DLTs:

1. Failure to meet treatment criteria day 8 (G2 ALT/AST)
2. Failure to meet treatment criteria day 8 (G3 neutropenia)
3. G3 ALT

### Dose-Delays:

1. Neutropenia
2. SBO/surgery
3. Viral respiratory infection (r/o covid)

### Dose-Reductions:

1. DL2→DL1 n=2 (G2 LFTs; G3 neutropenia; same DTL ots as above)
2. DL1 n=2 (neutropenia; fatigue)

The median time on treatment without progression was 330 days (range 42-617) with three patients continuing study at the time of data cutoff.

Median time to response was 81 days (range 43-207) and median duration of response was 169 days (range 126-364)

8 of the 10 patients were on treatment over 6 months; 4 out of 10 over 12 months, and 3 over 500 days.

Median time to partial response was 81 days (range 46-207).

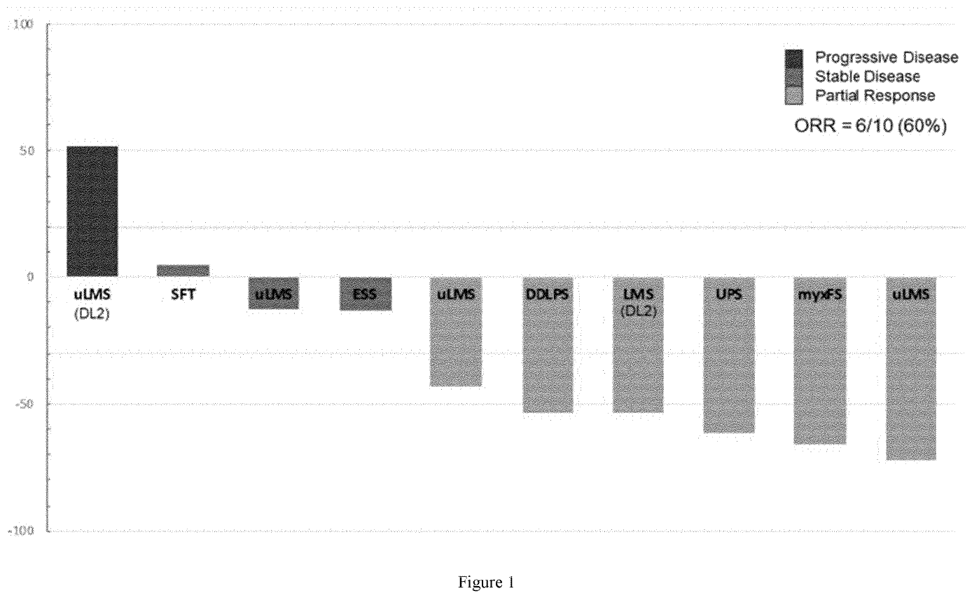

### Response Assessments (FIG. 1):

six patients have demonstrated RECIST 1.1 partial response in:
   undifferentiated pleomorphic sarcoma,
   leiomyosarcoma including uterine leiomyosarcoma.
   dedifferentiated liposarcoma,
   myxofibrosarcoma.
three patients have demonstrated RECIST 1.1 stable disease in:
   leiomyosarcoma (uterine leiomyosarcoma),
   endometrial stromal sarcoma,
   solitary fibrous tumor.

The recommended dose was determined to be lurbinectedin 3.2 mg/m² and doxorubicin 25 mg/m² on day 1 with a 21-day cycle.

### CONCLUSION

Full dose lurbinectedin with a low dose of doxorubicin is a feasible, well-tolerated and active combination. It achieved six partial responses and two prolonged stable disease (>430 days) in ten patients.

Overall, the present invention has identified exciting activity of lurbinectedin plus doxorubicin in the treatment of cancer. The present invention has identified dosing regimens which are particularly well-tolerated. Among other advantages, the regimens may allow higher number of cycles to be administered to the patient, prolonging the time of disease control. Moreover, the regimens may increase the response/disease control rate. In addition, the regimens may reduce the high levels of toxicity typically produced by doxorubicin.

1. Lurbinectedin for use in the treatment of cancer in a patient need thereof, wherein lurbinectedin is administered in combination with doxorubicin; and wherein doxorubicin is administered at a dose of 30 mg/m² or less during each administration cycle, or at a dose of less than 30 mg/m² during each administration cycle.

2. Lurbinectedin for use according to claim 1, wherein lurbinectedin is administered in combination with doxorubicin; and wherein doxorubicin is administered at a dose between 15 to 30 mg/m², or between 20 to 30 mg/m², or between 15 to less than 30 mg/m², or between 20 to less than 30 mg/m² during each administration cycle.

3. Lurbinectedin for use according to any of the previous claims, wherein lurbinectedin is administered in combination with doxorubicin; and wherein doxorubicin is administered at a dose between 15 to 25 mg/m², or between 20 to 25 mg/m², preferably about 25 mg/m², preferably 25 mg/m², during each administration cycle.

4. Lurbinectedin for use according to any of the previous claims, wherein doxorubicin is administered at a dose of about 25 mg/m².

5. Lurbinectedin for use according to any of the previous claims, wherein a single dose doxorubicin is administered at each administration cycle.

6. Lurbinectedin for use according to any of the previous claims, wherein lurbinectedin is administered at a dose of between 2 mg/m² to 4 mg/m², higher than 2 mg/m² and lower than 4 mg/m², between 2.5 and 3.5 mg/m², preferably about 3.2 mg/m², preferably 3.2 mg/m² body surface area during each administration cycle.

7. Lurbinectedin for use according to any of the previous claims, wherein lurbinectedin is administered at a dose of about 3.2 mg/m².

8. Lurbinectedin for use according to any of the previous claims, wherein the administration cycle is every three to four weeks, preferably every 21 days.

9. Lurbinectedin for use according to any of the previous claims, wherein lurbinectedin is administered on day 1 (D1) of each cycle; or wherein doxorubicin is administered on day 1 (D1) of each cycle; or wherein lurbinectedin and doxorubicin are administered on day 1 (D1) of each cycle.

10. Lurbinectedin for use according to any of the previous claims, wherein in combination with doxorubicin 1, 2, 3, 4, 5, 6, 7, 8, 9, 10, 11, 12, 13, 14, 15, 16, 17, 18 administration cycles are administered; preferably 2, 3, 4, 5, 6, 7, 8, 9, 10, 11, 12, 13, 14, 15, 16, 17, 18 administration cycles, preferably 7, 8, 9, 10, 11, 12, 13, 14, 15, 16, 17, 18 administration cycles.

11. Lurbinectedin for use according to any of the previous claims, wherein doxorubicin is administered first, followed by lurbinectedin administration.

12. Lurbinectedin for use according to any of the previous claims, wherein lurbinectedin is administered as 1 hour intravenous infusion during each administration cycle; or wherein doxorubicin is administered as 1 hour intravenous infusion during each administration cycle; wherein lurbinectedin and doxorubicin are administered as 1 hour intravenous infusion during each administration cycle.

13. Lurbinectedin for use according to any of the previous claims, wherein lurbinectedin is administered in combination with doxorubicin, wherein lurbinectedin is administered at a dose of about 3.2 mg/m² on D1 of each administration cycle; and wherein doxorubicin is administered at a dose of about 25 mg/m² administered on D1 of each administration cycle; and wherein each administration cycle is 21 days.

14. Lurbinectedin for use according to any of the previous claims, wherein the method further comprises administration of granulocyte-colony stimulating factor (G-CSF).

15. Lurbinectedin for use according to claim 14, wherein G-CSF is administered on day 1 of an administration cycle; or wherein the patient receives primary prophylaxis with G-CSF starting 24-72 hours after day 1 of an administration cycle, and for five days.

16. Lurbinectedin for use according to claim 14 or claim 15, wherein G-CSF is administered during the first administration cycle; optionally wherein G-CSF during one or more subsequent administration cycles.

17. Lurbinectedin for use according to any of the previous claims, wherein after combination administration of lurbinectedin and doxorubicin during a first phase; lurbinectedin is administered alone for one or more cycles during a second phase; wherein lurbinectedin may be administered according to one or more of claims 6 to 9, 12 or 14 to 16.

18. Lurbinectedin for use according to any of the previous claims, wherein the cancer is sarcoma, preferably a soft tissue sarcoma.

19. Lurbinectedin for use according to claim 18, wherein the soft tissue sarcoma is selected from from leiomyosarcoma, gastrointestinal stromal tumour, liposarcoma, dedifferentiated liposarcoma, undifferentiated pleomorphic sarcoma, malignant peripheral nerve sheath tumors, angiosarcoma, hemangioendothelioma, solitary fibrous tumor, fibrosarcoma, dermatofibrosarcoma protuberans, low-grade fibromyxoid sarcoma, endometrial stromal sarcoma, myxofibrosarcoma, fibromatosis, follicular dendritic cell sarcoma, desmoplastic small round cell tumour, pleomorphic liposarcoma, and synovial sarcoma.

20. Lurbinectedin for use according to claim 19, wherein the soft tissue sarcoma is advanced or metastatic leiomyosarcoma, selected from somatic soft tissue leiomyosarcoma, cutaneous or subcutaneous leiomyosarcoma, leiomyosarcoma of vascular origin, and leiomyosarcoma of uterine or non-uterine origin.

21. Lurbinectedin for use in the treatment of cancer in a patient need thereof, said treatment comprises:
i) administering to the patient one or more cycles of lurbinectedin in combination with doxorubicin in a first phase according to any one of claims 1 to 20, and
ii) administering to the patient one or more cycles of lurbinectedin alone in a second phase;
wherein lurbinectedin may be administered according to one or more of claims 6 to 9, 12 or 14 to 16.

22. A pharmaceutical package comprising lurbinectedin, together with instructions for its use in combination with doxorubicin according to any one of claims 1 to 21.
